(19) United States
(12) Patent Application Publication
(10) Pub. No.: US 2020/0290945 A1
(43) Pub. Date: Sep. 17, 2020

(54) Preparation and Purification of Biphenyldicarboxylic Acids

(71) Applicant: ExxonMobil Chemical Patents Inc., Baytown, TX (US)

(72) Inventors: Monica D. Lotz, Houston, TX (US); Etienne Mazoyer, Woluwe Saint Pierre (BE); Constantinos P. Bokis, The woodlands, TX (US); Javier Guzman, Porter, TX (US)

(21) Appl. No.: 16/753,056

(22) PCT Filed: Jun. 7, 2018
(86) PCT No.: PCT/US2018/036469

§ 371 Date: Apr. 2, 2020

Related U.S. Application Data
(60) Provisional application No. 62/589,696, filed on Nov. 22, 2017.
(60) Provisional application No. 62/589,658, filed on Nov. 22, 2017.

(51) Int. Cl. C07C 51/493; C07C 51/265; C07C 67/08; C07C 67/52

(52) U.S. Cl. CPC C07C 51/493; C07C 51/265; C08G 63/185; C07C 67/52; C07C 67/08

(57) ABSTRACT

Processes are described for purifying a biphenyldicarboxylic acid product containing one or more impurities, particularly at least formylbiphenylcarboxylic acid. In the processes, at least a portion of the biphenyldicarboxylic acid product is contacted with an alcohol under conditions effective to esterify at least part of the biphenyldicarboxylic acid and at least part of the formylbiphenylcarboxylic acid and produce an esterification effluent containing biphenyldicarboxylic acid diester and formylbiphenylcarboxylic acid ester. At least part of the biphenyldicarboxylic acid diester is then separated from the esterification effluent by crystallization. Advantageously, a polyester product may be produced from the separated biphenyldicarboxylic acid diester.

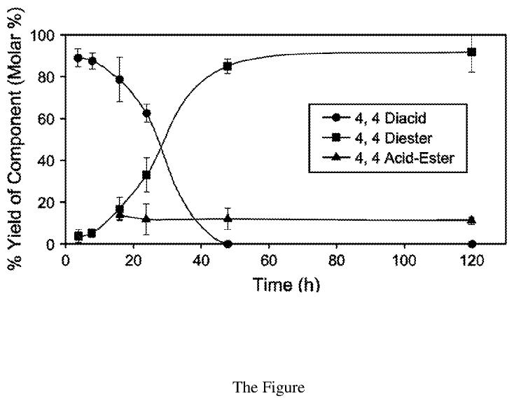

### CROSS-REFERENCE OF RELATED APPLICATIONS

This application claims the benefit of Provisional Application No. 62/589,696, filed Nov. 22, 2017, Provisional Application No. 62/589,658, filed Nov. 22, 2017, and PCT Application No. PCT/US2018/032210, filed May 11, 2018, the disclosures of which are incorporated herein by reference.

### FIELD

This disclosure relates to preparation and purification of biphenyldicarboxylic acids (BPDAs), and polyester products produced therefrom.

### BACKGROUND

BPDAs, especially the 3,4′ and 4,4′ isomers, are useful intermediates in the production of a variety of commercially valuable products, including polyesters and plasticizers for PVC and other polymer compositions. For example, BPDAs can be converted to ester plasticizers by esterification with a long chain alcohol. In addition, biphenyldicarboxylic acids are potential precursors, either alone or as a modifier for polyethylene terephthalate (PET), in the production of polyester fibers, engineering plastics, liquid crystal polymers for electronic and mechanical devices, and films with high heat resistance and strength. The 4,4′ isomer is the most desired for this service due to the advantageous properties of the resulting polymers.

As disclosed in U.S. Pat. Nos. 9,580,572 and 9,663,417, the entire disclosures of which are incorporated herein by reference in their entirety, BPDAs may be produced by hydroalkylation of toluene followed by dehydrogenation of the resulting (methylcyclohexyl)toluene (MCHT) to produce dimethylbiphenyl (DMBP) compounds. The DMBP compounds can then be oxidized to the desired diacids by reaction with an oxidant, such as oxygen, ozone or air, or any other oxygen source, such as hydrogen peroxide, in the presence of a catalyst, such as Co and/or Mn, at temperatures from 30° C. to 300° C.

Alternative routes via benzene are described in U.S. Pat. No. 9,085,669, the entire disclosure of which is incorporated herein by reference, in which the benzene is initially converted to biphenyl, either by oxidative coupling or by hydroalkylation to cyclohexyl benzene (CHB) followed by dehydrogenation of the CHB, and then the biphenyl is alkylated with methanol. The resultant DMBP compounds can then be oxidized to the desired diacids by the method described above.

One problem with existing routes to biphenyldicarboxylic acids through DMBP compounds is that the oxidation step, in addition to producing the desired diacids, also inevitably produces certain by-products, such as formylbiphenylcarboxylic acid (FBCA), that, if not removed, can (a) reduce polymer growth by chain termination when the BPDA is subsequently polymerized and (b) be detrimental to the color of the resultant polymer. Moreover, the nature of the BPDA (i.e., low volatility and formation of solid solutions) means that typical physical separation techniques (e.g., distillation and crystallization) are generally not effective in purifying the crude diacid.

There is, therefore, interest in developing alternative processes for purifying BPDAs produced by oxidation of DMBP compounds.

Other potential references of interest include U.S. Pat. No. 6,642,407; WO 2017/112031; and TW 201736432.

### SUMMARY

According to the present disclosure, it has now been found that a purified BPDA product can be produced from a crude product (typically obtained from the oxidation of DMBP) comprising (or consisting of, or consisting essentially of) BPDA and at least one acid impurity (typically formylbiphenyldicarboxylic acid (FBCA)), by reacting the crude product with an alcohol, such as methanol, to convert at least part of the BPDA and at least part of the at least one acid impurity, e.g., FBCA, to the corresponding esters. In particular, it has now been found that, although conventional distillation is ineffective in separating the individual esters, the BPDA diester can be recovered in high purity from the esterification effluent by crystallization. The recovered BPDA diester can be taken directly to polymerization or hydrolyzed to form purified diacid for subsequent polymerization.

Thus, in one aspect, the present disclosure provides a process for purifying a biphenyldicarboxylic acid product containing formylbiphenylcarboxylic acid, the process comprising (or consisting of, or consisting essentially of):

(a1) contacting at least a portion of the biphenyldicarboxylic acid product with an alcohol under conditions effective to esterify at least part of the biphenyldicarboxylic acid and at least part of the formylbiphenylcarboxylic acid and produce an esterification effluent containing biphenyldicarboxylic acid diester and formylbiphenylcarboxylic acid ester; and

(b1) separating at least part of the biphenyldicarboxylic acid diester from the esterification effluent by crystallization.

Typically, the biphenyldicarboxylic acid diester separated in (b1) may be hydrolyzed to produce biphenyldicarboxylic acid.

In a further aspect, the present disclosure provides a polyester product comprising the reaction product of a diol with a at least one of the following:

(i) the biphenyldicarboxylic acid diester separated in (b1); and/or

(ii) the biphenyldicarboxylic acid produced by hydrolyzing the biphenyldicarboxylic acid diester separated in (b1).

In a further aspect, the present disclosure provides a process for producing 3,4′- and/or 4,4′-biphenyldicaboxlyic acid diester, the process comprising (or consisting of, or consisting essentially of):

(a2) contacting 3,4′- and/or 4,4′-dimethylbiphenyl with a source of oxygen in the presence of an oxidation catalyst to produce an oxidation product comprising 3,4′- and/or 4,4′-biphenyldicarboxylic acid and one or more isomers of formylbiphenylcarboxylic acid;

(b2) contacting at least a portion of the oxidation product with an alcohol under conditions effective to esterify at least part of 3,4′- and/or 4,4′-biphenyldicarboxylic acid and at least part of the formylbiphenylcarboxylic acid and produce an esterification effluent containing 3,4′- and/or 4,4′-biphenyldicarboxylic diester and formylbiphenylcarboxylic acid ester; and

(c2) separating at least part of the 3,4′- and/or 4,4′-biphenyldicarboxylic diester from the esterification effluent by crystallization.

### BRIEF DESCRIPTION OF THE DRAWING

The FIGURE is a graph plotting the molar concentration against time on stream for various components in the reaction effluent produced by the esterification of 4,4′-biphenyl dicarboxylic acid with methanol according to Example 1.

### DETAILED DESCRIPTION OF THE PREFERRED EMBODIMENTS

As used herein, “wt %” means percentage by weight, and “ppm wt” and “wppm” are used interchangeably to mean parts per million on a weight basis. All “ppm” as used herein are ppm by weight unless specified otherwise. All concentrations herein are expressed on the basis of the total amount of the composition in question. Thus, the concentrations of the various components of the first mixture are expressed based on the total weight of the first mixture. All ranges expressed herein should include both end points as two specific embodiments unless specified or indicated to the contrary.

Unless otherwise indicated, room temperature is 23° C.

Nomenclature of elements and groups thereof used herein are pursuant to the Periodic Table used by the International Union of Pure and Applied Chemistry after 1988. An example of the Periodic Table is shown in the inner page of the front cover of Advanced Inorganic Chemistry, 6th Edition, by F. Albert Cotton et al. (John Wiley & Sons, Inc., 1999).

The present disclosure relates to processes for producing a purified biphenyldicarboxylic acid (BPDA) product from a crude product comprising BDPA and at least one acid impurity, typically at least formylbiphenylcarboxylic acid (FBCA). Preferably, the crude product is derived from the catalytic oxidation of at least one dimethylbiphenyl (DMBP) compound. The purification processes described herein typically comprise contacting at least a portion of the crude BPDA product with an alcohol under conditions effective to esterify at least part of the BPDA and at least part of the at least one acid impurity, to e.g., FBCA, in the product portion and produce an esterification effluent containing biphenyldicarboxylic acid diester and an aldehyde-ester formed from the at least one acid impurity, e.g., formylbiphenylcarboxylic acid ester. At least part of the biphenyldicarboxylic acid diester is then separated from the esterification effluent by crystallization. It has presently been found that, whereas modeling data suggest that conventional distillation, even with a large number of stages, is ineffective in separating the BPDA acid diester from the aldehyde-ester, crystallization offers a viable path for obtaining substantially pure BPDA diester from the esterification effluent. Generally, crystallization is further effective in removing any remaining amounts of color producing bodies from the BPDA acid diester, particularly residual amounts of catalyst and/or promoter. The recovered BPDA diester can be taken directly to polymerization or hydrolyzed to form purified diacid for subsequent polymerization.

### Production of Biphenyldicarboxylic Acid

As discussed above, the present processes are particularly directed towards the purification of a crude product (typically obtained from the oxidation, preferably catalytic oxidation, of DMBP) comprising BPDA and at least one acid impurity (typically FBCA). Any known method can be used to produce the DMBP starting material but, in one or more embodiments, a preferred process initially involves the conversion of toluene to (methylcyclohexyl)toluene (MCHT) in the presence of hydrogen over a hydroalkylation catalyst according to the following reaction:

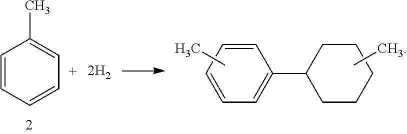

The MCHT can then be dehydrogenated to produce the desired DMBP product.

The catalyst employed in the hydroalkylation reaction is generally a bifunctional catalyst comprising a hydrogenation component and a solid acid alkylation component, typically a molecular sieve. The catalyst may also include a binder such as clay, alumina, silica and/or metal oxides. The latter may be either naturally occurring or in the form of gelatinous precipitates or gels including mixtures of silica and metal oxides. Naturally occurring clays which can be used as a binder include those of the montmorillonite and kaolin families, which families include the subbentonites and the kaolins commonly known as Dixie, McNamee, Georgia and Florida clays or others in which the main mineral constituent is to halloysite, kaolinite, dickite, nacrite or anauxite. Such clays can be used in the raw state as originally mined or initially subjected to calcination, acid treatment or chemical modification. Suitable metal oxide binders include silica, alumina, zirconia, titania, silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

Any known hydrogenation metal or compound thereof can be employed as the hydrogenation component of the catalyst, although suitable metals include palladium, ruthenium, nickel, zinc, tin, and cobalt, with palladium being particularly advantageous. In certain embodiments, the amount of hydrogenation metal present in the catalyst is between 0.05 and 10 wt %, more particularly from 0.1 and 5 wt %, of the catalyst.

Often, the solid acid alkylation component comprises a large pore molecular sieve having a Constraint Index (as defined in U.S. Pat. No. 4,016,218) less than 2. Suitable large pore molecular sieves include zeolite beta, zeolite Y, Ultrastable Y (USY), Dealuminized Y (Deal Y), mordenite, ZSM-3, ZSM-4, ZSM-18, and ZSM-20. Zeolite ZSM-4 is described in U.S. Pat. No. 4,021,447. Zeolite ZSM-20 is described in U.S. Pat. No. 3,972,983. Zeolite Beta is described in U.S. Pat. No. 3,308,069, and Re. No. 28,341. Low sodium Ultrastable Y molecular sieve (USY) is described in U.S. Pat. Nos. 3,293,192 and 3,449,070. Dealuminized Y zeolite (Deal Y) may be prepared by the method found in U.S. Pat. No. 3,442,795. Zeolite UHP-Y is described in U.S. Pat. No. 4,401,556. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

More preferably, the solid acid alkylation component comprises a molecular sieve of the MCM-22 family. The term “MCM-22 family material” (or “material of the MCM-22 family” or “molecular sieve of the MCM-22 family”), as used herein, includes one or more of: molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference); molecular sieves made from a common second degree building block, being a 2-dimensional tiling of such MWW framework topology unit cells, forming a monolayer of one unit cell thickness, preferably one c-unit cell thickness; molecular sieves made from common second degree building blocks, being layers to of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and molecular sieves made by any regular or random 2-dimensional or 3-dimensional combination of unit cells having the MWW framework topology.

Molecular sieves of MCM-22 family generally have an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as the incident radiation and a diffractometer equipped with a scintillation counter and associated computer as the collection system. Molecular sieves of MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO 97/17290), MCM-36 (described in U.S. Pat. No. 5,250,277), MCM-49 (described in U.S. Pat. No. 5,236,575), MCM-56 (described in U.S. Pat. No. 5,362,697) and mixtures thereof.

In addition to the toluene and hydrogen, the feed to the hydroalkylation reaction may include benzene and/or xylene which can undergo hydroalkylation to produce various methylated cyclohexylbenzene molecules having 12 to 16 carbon atoms. A diluent, which is substantially inert under hydroalkylation conditions, may also be included in the hydroalkylation feed. In certain embodiments, the diluent is a hydrocarbon, in which the desired cycloalkylaromatic product is soluble, such as a straight chain paraffinic hydrocarbon, a branched chain paraffinic hydrocarbon, and/or a cyclic paraffinic hydrocarbon. Examples of suitable diluents are decane and cyclohexane. Although the amount of diluent is not narrowly defined, desirably the diluent is added in an amount such that the weight ratio of the diluent to the aromatic compound is at least 1:100; for example at least 1:10, but no more than 10:1, desirably no more than 4:1.

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are from 100° C. to 400° C., more particularly from 125° C. to 250° C., while suitable reaction pressures are between 100 and 7,000 kPa, more particularly from 500 and 5,000 kPa. The molar ratio of hydrogen to aromatic feed is typically from 0.15:1 to to 15:1.

It has been found that MCM-22 family molecular sieves are particularly active and stable catalysts for the hydroalkylation of toluene. In addition, catalysts containing MCM-22 family molecular sieves exhibit improved selectivity to the 3,3′-dimethyl, the 3,4′-dimethyl, the 4,3′-dimethyl and the 4,4′-dimethyl isomers in the hydroalkylation product, while at the same time reducing the formation of fully saturated and heavy by-products. For example, using an MCM-22 family molecular sieve with a toluene feed, it is found that the hydroalkylation reaction product may comprise: at least 60 wt %, preferably at least 70 wt %, for example at least 80 wt % of the 3,3′, 3,4′, 4,3′ and 4,4′-isomers of MCHT based on the total weight of all the MCHT isomers; less than 40 wt %, preferably less than 30 wt %, for example from 15 to 25 wt % of the 2,2′, 2,3′, and 2,4′-isomers of MCHT based on the total weight of all the MCHT isomers; less than 30 wt % of methylcyclohexane and less than 2% of dimethylbicyclohexane compounds; and less than 1 wt % of compounds containing in excess of 14 carbon atoms, such as di(methylcyclohexyl)toluene.

The hydroalkylation reaction product may also contain significant amounts of residual toluene, for example up to 50 wt %, preferably up to 90 wt %, typically from 60 to 80 wt % of residual toluene based on the total weight of the hydroalkylation reaction product. The residual toluene can readily be removed from the reaction effluent by, for example, distillation. The residual toluene can then be recycled to the hydroalkylation reactor, together with some or all of any unreacted hydrogen. In some embodiments, it may be desirable to remove the C₁₄₊ reaction products, such as di(methylcyclohexyl)toluene, by distillation.

The remainder of the hydroalkylation reaction effluent, composed mainly of (methylcyclohexyl)toluenes, is then dehydrogenated to convert the (methylcyclohexyl)toluenes to the corresponding methyl-substituted biphenyl compounds. The dehydrogenation is conveniently conducted at a temperature from 200° C. to 600° C. and a pressure from 100 kPa to 3550 kPa (atmospheric to 500 psig) in the presence of dehydrogenation catalyst. A suitable dehydrogenation catalyst comprises one or more elements or compounds thereof selected from Group 10 of the Periodic Table of Elements, for example platinum, on a support, such as silica, alumina or carbon nanotubes. Often, the Group 10 element is present in an amount from 0.1 to 5 wt % of the catalyst. In some cases, the dehydrogenation catalyst may also include tin or a tin compound to improve the selectivity to the desired methyl-substituted biphenyl product. Often, the tin is present in an amount from 0.05 to 2.5 wt % of the catalyst.

Particularly using an MCM-22 family-based catalyst for the upstream to hydroalkylation reaction, the product of the dehydrogenation step comprises DMBP compounds in which the concentration of the 3,3′-, 3,4′- and 4,4′ isomers is at least 50 wt %, preferably at least 60 wt %, for example at least 70 wt % based on the total weight of DMBP compounds. Typically, the concentration of the 2,X′-DMBP isomers in the dehydrogenation product is less than 50 wt %, preferably less than 30 wt %, for example from 5 to 25 wt % based on the total weight of DMBP compounds.

In other embodiments of the present processes, the DMBP starting material can be produced from benzene via conversion of the benzene to diphenyl followed by methylation of the biphenyl to DMBP. For example, it is known that benzene can be converted directly to biphenyl by reaction with oxygen over an oxidative coupling catalyst as follows:

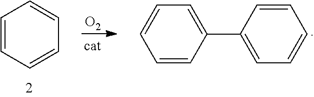

Details of the oxidative coupling of benzene can be found in Ukhopadhyay, Sudip; Rothenberg, Gadi; Gitis, Diana; Sasson, Yoel, Casali Institute of Applied Chemistry, Hebrew University of Jerusalem, Israel, Journal of Organic Chemistry (2000), 65(10), pp. 3107-3110, incorporated herein by reference.

Alternatively, benzene can be converted to biphenyl by hydroalkylation to cyclohexylbenzene according to the reaction:

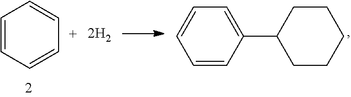 [SMILES: c1ccccc1 ; c1ccc(C2CCCCC2)cc1]

followed by dehydrogenation of the cyclohexylbenzene.

In such a process, the benzene hydroalkylation can be conducted in the same manner as described above for the hydroalkylation of toluene, while the dehydrogenation of the cyclohexylbenzene can be conducted in the same manner as described above for the dehydrogenation of (methylcyclohexyl)toluene.

In either case, the biphenyl product of the oxidative coupling step or the hydroalkylation/dehydrogenation sequence is then methylated, for example with methanol, to produce DMBP. Any known alkylation catalyst can be used for the methylation reaction, such as an intermediate pore molecular sieve having a Constraint Index (as defined in U.S. Pat. No. 4,016,218) of 3 to 12, for example ZSM-5.

The composition of the methylated product will depend on the catalyst and to conditions employed in the methylation reaction, but inevitably will comprise a mixture of the different isomers of DMBP. Typically, the methylated product will contain from 50 to 100 wt % of 3,3′-, 3,4′- and 4,4′-DMBP isomers and from 0 to 50 wt % of 2,X′ (where X′ is 2′, 3′ or 4′)-DMBP isomers based on the total weight of DMBP compounds in the methylation product.

Irrespective of the process used to produce the DMBP starting material, the raw dimethylbiphenyl product from the production sequences described above will contain unreacted components and by-products in addition to a mixture of DMBP isomers. For example, where the initial feed comprises toluene and the production sequence involves hydroalkylation to MCHT and dehydrogenation of the MCHT, the raw DMBP product will tend to contain residual toluene and MCHT as well as by-products including hydrogen, methylcyclohexane dimethylcyclohexylbenzene, and C₁₅₊ heavy hydrocarbons in addition to the target DMBP isomers. Thus, in some embodiments, prior to any separation of the DMBP isomers, the raw product of the MCHT dehydrogenation is subjected to a rough cut separation to remove at least part of the residues and by-products with significantly different boiling points from the DMBP isomers. For example, the hydrogen by-product can be removed and recycled to the hydroalkylation and/or MCHT dehydrogenation steps, while residual toluene and methylcyclohexane by-product can be removed and recycled to the hydroalkylation step. Similarly, part of the heavy (C₁₅₊) components can be removed in the rough cut separation and can be recovered for use as a fuel or can be reacted with toluene over a transalkylation catalyst to convert some of the dialkylate to additional MCHT. A suitable rough cut separation can be achieved by distillation. For example, the H₂ and C₇ components can be stripped from the C₁₂₊ components without reflux.

After partial removal of the by-products and residual components in the rough cut separation, the remaining dimethylbiphenyl product is typically subjected to a first DMBP separation step, in which the product is separated into at least a first stream rich in 3,4′ and 4,4′ DMBP and at least one second stream comprising one or more 2,X′ (where X′ is 2′, 3′, or 4′) and 3,3′ DMBP isomers. The second stream will also typically contain most of the unreacted MCHT and most of the dimethylcyclohexylbenzene by-product in the raw dimethylbiphenyl product. A suitable process for effecting this initial separation is crystallization and/or distillation operating below or at, atmospheric pressure.

In certain embodiments, part or all of the first stream can be recovered and, optionally after further purification, can be oxidized to produce BPDA as described below. Additionally or alternatively, part or all of the first stream may be subjected to a second to DMBP separation step to separate the first stream into a third stream rich in 4,4′ DMBP and a fourth stream comprising 3,4′ DMBP. Because of the differences in fusion temperatures of these isomers, the second DMBP separation is conveniently effected by fractional crystallization. In some embodiments, the fractional crystallization is assisted by the addition of a solvent, preferably a C₃ to C₁₂ aliphatic hydrocarbon, more preferably pentane and/or hexane, to the first stream. Either or both of the third and fourth streams can then be oxidized to produce BPDA as described below.

In some embodiments the second stream, 2,X′ (where X′ is 2′, 3′, or 4′) and 3,3′ DMBP isomers, can undergo further treatment, such as isomerization to increase the concentration of the more desirable 3,4′, and especially 4,4′, isomer.

Any of the DMBP isomer-containing streams described above can be oxidized to produce the corresponding biphenyldicarboxylic acid. Preferably, the DMBP isomer-containing stream is rich in 3,4′ and/or 4,4′ DMBP, such as the first, third, and fourth streams described above. Particularly preferably, the DMBP isomer-containing stream is rich in 4,4′ DMBP, such as the third stream described above. For example, the DMBP isomer-containing stream preferably comprises at least 50 wt % of 3,4′ and/or 4,4′ DMBP (more preferably 4,4′ DMBP), preferably at least 75 wt %, or at least 90 wt % of 3,4′ and/or 4,4′ DMBP (more preferably 4,4′ DMBP). The oxidation can be performed by any process known in the art, such as via thermal or catalytic methods. Preferably, the oxidation can be performed by reacting the methyl-substituted biphenyl compounds with an oxidant, such as oxygen, ozone or air, or any other oxygen source, such as hydrogen peroxide, in the presence of a catalyst and with or without a promoter, such as Br, at temperatures from 30° C. to 300° C., more particularly from 60° C. to 200° C. Suitable catalysts comprise Co or Mn or a combination of both metals, such as cobalt acetate or cobalt (II) chloride hexahydrate. The oxidation is normally conducted with the DMBP isomer(s) being in solution, generally in acetic acid as solvent. Generally, in such aspects BPDA is formed in a solid state within the solution.

### Purification of Biphenylcarboxylic (BPDA) Product

Typically, the crude product from the oxidation of DMBP will contain 50 wt % or more, preferably 75 wt % or more, more preferably 90 wt % or more, and ideally 95 wt % or more of BPDA. The relative amounts of the BPDA isomers in the crude product will vary depending on the composition of the oxidized DMBP stream. For example, in the oxidation of a 4,4′ DMBP rich stream, 4,4′ BPDA will be the predominant BPDA isomer, e.g., greater than 50 wt % of the total weight BPDA compounds in the crude product. Typically, the crude product will contain from 50 to 100 wt % of 3,4′- and/or 4,4′-BPDA isomers and from 0 to to 50 wt % of 3,3′ and/or 2,X′ (where X′ is 2′, 3′ or 4′)-BPDA isomers based on the total weight of BPDA compounds in the crude product.

However, in addition to the desired BPDA, the crude product from the oxidation of DMBP will inherently contain certain impurities, such as side products, intermediates, and residual oxidation catalyst. Generally, the impurities contain under-oxidized species, especially one or more isomers of formylbiphenylcarboxylic acid (FBCA) and/or biphenylmonocarboxylic acid, as illustrated by the following formulas:

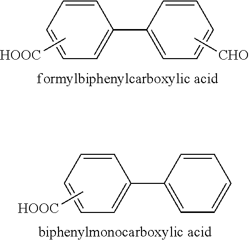

which can be present in levels as high as 50 wt %, more commonly 20 wt % or less, such as 10 wt % or less, or 5 wt % or less, or 1 wt % or less, of the crude oxidation product, together with some quinone and fluorenone derivatives. Similarly to the desired BPDA, the relative amounts of the FBCA isomers in the crude product will vary depending on the composition of the oxidized DMBP stream. For example, in the oxidation of a 4,4′ DMBP rich stream, 4,4′ FBCA will be the predominant FBCA isomer, e.g., greater than 50 wt % based on the total weight of FBCA compounds in the crude product. Typically, the crude product will contain from 50 to 100 wt % of 3,4′- and/or 4,4′-FBCA isomers and from 0 to 50 wt % of 3,3′- and/or 2,X′ (where X′ is 2′, 3′ or 4′)-FBCA isomers based on the total weight of FBCA compounds in the crude product.

Most of the aforementioned impurities are not desired in the finished product as they will induce formation low molecular weight (by chain termination) species and detrimental color when the BPDA is polymerized. Removal or reduction in concentration of these impurities is therefore required, but common separation methods are generally ineffective. For example, 3,4′ and 4,4′-BPDA each have extremely reduced volatility which makes purification via distillation impractical. Additionally, it has been found that, as a result of their common geometry, the impurity 4,4′-FBCA forms a solid solution with 4,4′-BPDA. Similarly, it is further expected that the impurity 3,4′-FBCA forms a solid solution with 3,4′-BPDA. Accordingly, purification via crystallization is not effective either. To obviate or reduce these problems, the present processes include the additional steps of reacting at least part of the crude BPDA product with an alcohol under conditions effective to esterify the acid species present in the crude product and produce an esterification effluent containing biphenyldicarboxylic acid diester, together with formylbiphenylcarboxylic acid ester and, in to some cases biphenylmonocarboxylic acid ester. The esterification effluent may then be subjected to crystallization to separate the biphenyldicarboxylic acid diester at greater than 99 wt % purity.

The alcohol employed in the esterification step is not critical, but generally alcohols having 1 to 20 carbons atoms, such as methanol and ethanol, and especially methanol, are preferred. The conditions employed in the esterification step are similarly not critical but suitable conditions comprise a temperature from 25° C. to 330° C., more particularly from 200° C. to 330° C. To maximize production of dicarboxylic acid diester, the molar ratio of alcohol relative to the acid species in the crude BPDA product should generally be greater than 2, preferably greater than 10.

The esterification reaction can be conducted with or without a catalyst, although reaction times are generally shorter when a catalyst is employed. For example, without a catalyst, reaction times of 4 to 120 hours are typically required. Most preferably the time on stream (reaction time) of the inventive process (without catalyst) is at least 6, or 8, or 10 hours, or within a range from 6, or 8, or 10, or 12, or 16 hours to 40, or 45, or 50, or 60 hours. In addition to the desired biphenyldicarboxylic acid diester (“diester”), the esterification reaction product will typically inherently contain some amount of under-esterified species, i.e., biphenylmonocarboxylic acid ester (“acid-ester”). For example, in the esterification of 4,4′-biphenyl dicarboxylic acid with methanol, the esterification effluent will generally contain biphenyl-4,4′-dicarboxylic acid monomethylester in addition to the desired 4,4′-dicarboxylic acid dimethylester product. For example, the esterification effluent may contain from 5 mol % to 15 mol % of the acid-ester, such as 12 mol %. At shorter reaction times, reactions with added catalyst result in significantly higher conversion of diacid. Suitable catalysts comprise solid acid catalysts, such as ion exchange resins, such as amberlyst, and heteropoly acids, as well as dissolved inorganic acid and organic acid catalysts, such as sulfuric acid and p-toluene sulfonic acid.

The esterification reaction produces water as a byproduct. Accordingly, a water removal process is typically employed. The water removal process may be performed during the esterification reaction or in a post-esterification separation step. Additionally, a post-esterification separation step is typically employed to remove catalyst in aspects where the esterification is conducted with a catalyst. For example, the esterification product can be cooled to form a precipitate. The precipitate can be filtered to recover the precipitate from a majority of the water and catalyst. The recovered precipitate can be washed with solvent to further remove catalyst and subsequently dried to remove water.

After esterification, and optionally catalyst and/or water removal, is complete, the effluent is fed to a crystallizer, where the biphenyldicarboxylic acid diester selectively crystallizes out of the effluent to form a purified BPDA diester product and a crystallizer mother liquor comprising the separated formylbiphenylcarboxylic acid diester and optionally, trace amounts of catalyst (e.g., oxidation catalyst) and/or promoter. Crystallization can be conducted at or above room temperature, but preferably is conducted at a temperature from 50° C. to 200° C., more particularly from 75° C. to 150° C. In this way, from a crude biphenyldicarboxylic acid product containing at least 0.05 wt % of formylbiphenylcarboxylic acid, it is possible to separate a purified biphenyldicarboxylic acid diester containing less than 200 ppmw of formylbiphenylcarboxylic acid ester, preferably less than 20 ppmw of formylbiphenylcarboxylic acid.

The purified BPDA diester can then be fed directly to polymerization or hydrolyzed to form purified diacid for subsequent polymerization. Particularly preferably, polyesters may be prepared from the purified BPDA diester or diacid, ether by conventional direct esterification or transesterification methods. Suitable diols for reaction with the above-mentioned diester or diacid compositions include alkanediols having 2 to 12 carbon atoms, such as monoethylene glycol, diethylene glycol, 1,3-propanediol, or 1,4-butane diol, 1,6-hexanediol, and 1,4-cyclohexanedimethanol. Optionally, the BPDA diester or diacid compositions may be further reacted with terephthalic acid or terephthalate. Suitable catalysts include but are not limited to titanium alkoxides such as titanium tetraisopropoxide, dialkyl tin oxides, antimony trioxide, manganese (II) acetate and Lewis acids. Suitable conditions include a temperature from 170 to 350° C. for a time from 0.5 hours to 10 hours. Generally, the reaction is conducted in the molten state and so the temperature is selected to be above the melting point of the monomer mixture but below the decomposition temperature of the polymer. A higher reaction temperature is therefore needed for higher percentages of biphenyl dicarboxylic acid in the monomer mixture. The polyester may be first prepared in the molten state followed by a solid state polymerization to increase its molecular weight or intrinsic viscosity for applications like bottles.

The invention will now be more particularly described with reference to the following non-limiting Examples.

### EXAMPLES

### HPLC Method

In Example 1, high performance liquid chromatography (HPLC) was performed using the following procedure. Samples were analyzed on an Agilent Technologies 1100 to Series system equipped with a Phenomenex™ Synergi Hydro-RP phase column (100×2 mm inner diameter and 2.5 μm particles) and DAD detector (254 and 280 nm). The HPLC was performed with at room temperature (23° C.) with an eluent rate of 0.4 ml/min. The mobile phase was 80/20 water (0.1% formic acid)/ACN for the initial 10 minutes, ramped to 35/65 water (0.1% formic acid)/ACN over a period of 50 minutes, ramped to 15/85 water (0.1% FA)/ACN over a period of 10 minutes. The response factor of the different components were determined using <200 ppm dilutions in dimethyl acetamide (DMA).

### Example 1: Esterification

A series of batch experiments were performed to study the thermal esterification of 4,4′-biphenyl dicarboxylic acid without catalyst with methanol at various reaction times. Each of the experiments was carried out by loading a solution of 12 wt % 4,4′-biphenyl dicarboxylic acid in methanol into a 150 mL stainless steel Parr reactor, pressurizing to 600 psig (4238 kPa-a) with nitrogen and heating to a reaction temperature of 220° C. The components in the resulting effluent at a variety of reaction times were then measured using HPLC.

The results of these experiments summarized in The FIGURE, which depicts the molar concentration against time on stream for various components in the reaction effluent in the absence of catalyst. It will be seen from The FIGURE that conversion starts to significantly increase after 8 hours, until nearly complete conversion is reached at 48 hours. As also seen from The FIGURE, the yield of diester (i.e., dicarboxylic acid dimethylester) with >99% conversion of diacid starting material appears to be limited to 85% under these conditions, accompanied by 12% yield of acid-ester (i.e., biphenyl-4,4′-dicarboxylic acid monomethylester).

### Examples 2-3: Distillation (Comparative) and Crystallization

In Examples 2 and 3, a suite of thermodynamic data on 4,4′-substituted biphenyl components was generated and used to model the results of both distillation and crystallization of a mixture comprising 96 wt % 4,4′-biphenyldicarboxylic acid dimethylester (BP44DME), 2 wt % 4,4′-formylbiphenylcarboxylic acid monomethylester (BP4A4ME) and 2 wt % 4,4′-biphenyldicarboxylic acid monomethylester (4MBP4ME).

### Comparative Example 2: Distillation

In Comparative Example 2, the composition of the overhead and bottom streams in a 30-stage distillation at 10 mm Hg of the mixture described above was modeled, wherein the modeled distillation comprised a feed temperature of 240° C. and a reflux ratio of 5. In order to model distillation behavior, (1) volatility (vapor pressure) information for each species and (2) binary vapor-liquid equilibrium (VLE) information for each binary pair of species were first generated. A thermodynamic model mimicking the generated volatility and VLE information was then applied and used to model the distillation in a process simulation tool. The thermodynamic model chosen was non-random two-liquid model (NRTL) and the process simulation tool was Aspen Plus™ version 8.8.

Accordingly, for the three 4,4′-substituted biphenyl components of the simulation mixture, the following values were generated:
BP44DME: vapor pressure values (measured experimentally);
BP4A4ME: vapor pressure values (measured experimentally);
4MBP4ME: vapor pressure values (estimated in Aspen Plus using the Riedel vapor correlation model);
BP44DME+BP4A4ME binary: VLE values (measured experimentally);
BP44DME+4MBP4ME binary: VLE data (predicted by group contribution method UNIFAC); and
BP4A4ME+4MBP4ME binary: VLE data (predicted by group contribution method UNIFAC).

NRTL interaction parameters for each binary pair were estimated from the above information and are entered together with the vapor pressure values into Aspen Plus to model the distillation scenario.

The models indicate that a 30-stage distillation at 10 mm Hg of the mixture described above with a feed temperature of 240° C. and a reflux ratio of 5 produces (a) an overhead stream containing 20 wt % of the distillate composed of 87.2 wt % 4,4′-biphenyldicarboxylic acid diester, 2.8 wt % 4,4′-formylbiphenylcarboxylic acid ester and 10 wt % 4,4′-biphenyldicarboxylic acid monoester; and (b) a bottom stream containing 80 wt % of the distillate composed of 98.2 wt % 4,4′-biphenyldicarboxylic acid diester and 1.8 wt % 4,4′-formylbiphenylcarboxylic acid ester.

### Example 3: Crystallization

In Example 3, the composition of the crystalline product and mother liquor in the crystallization of the mixture described above performed at 112° C. and ambient pressure was modeled. In order to model crystallization, the melting temperature and melting enthalpy of each crystallizing species were generated, as well as binary solid-liquid equilibrium (SLE) values for each binary pair of crystallizing species to model interaction behavior of these binary pairs with the liquid phase of the solution (e.g., liquid phase non-idealities, or activity coefficients). A thermodynamic model mimicking the generated information was then applied to and used to generate the SLE equations governing the modeled crystallization. The thermodynamic model chosen was non-random two-liquid model (NRTL). For the purposes of the example, it is assumed that 4MBP4ME will not crystallize out of the solution. Accordingly, for the two crystallizing species of the simulation mixture, the following values were generated:
BP44DME: melting temperature (216.9° C.) and melting enthalpy (42568 J/Kmol), each measured experimentally via calorimetry;
BP4A4ME: melting temperature (118.4° C.) and melting enthalpy (21150 J/Kmol), each measured experimentally via calorimetry;
BP44DME+BP4A4ME binary: SLE values (predicted by solid solubility thermodynamic theory combined with group contribution method UNIFAC); and
BP4A4ME+4MBP4ME binary: SLE values (predicted by solid solubility thermodynamic theory combined with group contribution method UNIFAC).

NRTL interaction parameters for each binary pair were estimated from the above data and are input together with the experimentally determined melting temperature and enthalpy data into a MATLAB™ program configured to perform the SLE calculations that govern crystallization.

The models indicate that crystallization at 112° C. separates the mixture into (a) a crystalline product containing 95 mol % of the total crystallization product composed of 100 mol % 4,4′-biphenyldicarboxylic acid diester and (b) a mother liquor containing 5 mol % of the total crystallization product composed of 9 mol % 4,4′-biphenyldicarboxylic acid diester, 47 mol % 4,4′-formylbiphenylcarboxylic acid ester, and 44 mol % 4,4′-biphenyldicarboxylic acid monoester.

While the present invention has been described and illustrated by reference to particular embodiments, those of ordinary skill in the art will appreciate that the invention lends itself to variations not necessarily illustrated herein. All documents described herein are incorporated by reference herein, including any priority documents and/or testing procedures to the extent they are not inconsistent with this text.

1. A process for purifying a biphenyldicarboxylic acid product containing biphenyldicarboxylic acid and formylbiphenylcarboxylic acid, the process comprising:
(a1) contacting at least a portion of the biphenyldicarboxylic acid product with an alcohol under conditions effective to esterify at least part of the biphenyldicarboxylic acid and at least part of the formylbiphenylcarboxylic acid and produce an esterification effluent containing biphenyldicarboxylic acid diester and formylbiphenylcarboxylic acid ester; and
(b1) separating at least part of the biphenyldicarboxylic acid diester from the esterification effluent by crystallization.

2. The process of claim 1, wherein the biphenyldicarboxylic acid is 3,4′-biphenyldicarboxylic acid, 4,4′-biphenyldicarboxylic acid or a mixture thereof.

3. The process of claim 1, wherein the biphenyldicarboxylic acid product is derived from the catalytic oxidation of at least one dimethylbiphenyl compound.

4. The process of claim 1, wherein the conditions in the contacting step (a1) comprise a temperature from 25° C. to 300° C.

5. The process of claim 1, wherein the contacting (a1) is conducted in the absence of a catalyst.

6. The process of claim 1, wherein the contacting (a1) is conducted in the presence of a catalyst.

7. The process of claim 6, wherein the catalyst comprises at least one of a solid acid catalyst and a dissolved inorganic acid and organic acid catalyst.

8. The process of claim 1, wherein the alcohol comprises at least one alcohol having 1 to 20 carbons atoms.

9. The process of claim 8, wherein the alcohol comprises methanol.

10. The process of claim 1, wherein the crystallization is conducted at a temperature from 50° C. to 200° C.

11. The process of claim 1, wherein the biphenyldicarboxylic acid product contains at least 0.05 wt % of formylbiphenylcarboxylic acid and the biphenyldicarboxylic acid diester separated in (b1) contains less than 200 ppmw of formylbiphenylcarboxylic acid ester.

12. The process of claim 11, wherein the biphenyldicarboxylic acid diester separated in (b1) contains less than 20 ppmw of formylbiphenylcarboxylic acid ester.

13. The process of claim 1, and further comprising hydrolyzing the biphenyldicarboxylic acid diester separated in (b1) to produce biphenyldicarboxylic acid.

14. A polyester product comprising the reaction product of a diol with at least one of the following:
(i) the biphenyldicarboxylic acid diester separated in (b1) according to claim 1; and/or
(ii) the biphenyldicarboxylic acid produced according to claim 13.

15. A process for producing 3,4′- and/or 4,4′-biphenyldicarboxylic acid diester, the process comprising:
(a2) contacting 3,4′- and/or 4,4′-dimethylbiphenyl with a source of oxygen in the presence of an oxidation catalyst to produce an oxidation product comprising 3,4′- and/or 4,4′-biphenyldicarboxylic acid and one or more isomers of formylbiphenylcarboxylic acid;
(b2) contacting at least a portion of the oxidation product with an alcohol under conditions effective to esterify at least part of 3,4′- and/or 4,4′-biphenyldicarboxylic acid and at least part of the formylbiphenylcarboxylic acid and produce an esterification effluent containing 3,4′- and/or 4,4′-biphenyldicarboxylic diester and formylbiphenylcarboxylic acid ester; and
(c2) separating at least part of the 3,4′- and/or 4,4′-biphenyldicarboxylic diester from the esterification effluent by crystallization.

16. The process of claim 15, wherein the conditions in the contacting step (b2) comprise a temperature from 25° C. to 300° C.

17. The process of claim 15, wherein the contacting (b2) is conducted in the absence of a catalyst.

18. The process of claim 15, wherein the contacting (b2) is conducted in the presence of a catalyst.

19. The process of claim 18, wherein the catalyst comprises at least one of a solid acid catalyst and a dissolved inorganic acid and organic acid catalyst.

20. The process of claim 15, wherein the alcohol comprises at least one alcohol having 1 to 20 carbons atoms.

21. The process of claim 20, wherein the alcohol comprises methanol.

22. The process of claim 15, wherein the crystallization is conducted at a temperature from 50° C. to 200° C.

23. The process of claim 15, wherein the oxidation product contains at least 0.05 wt % of formylbiphenylcarboxylic acid and the 3,4′- and/or 4,4′-biphenyldicarboxylic diester separated in (c2) contains less than 200 ppmw of formylbiphenylcarboxylic acid ester.

24. The process of claim 23, wherein the biphenyldicarboxylic acid diester separated in (c2) contains less than 20 ppmw of formylbiphenylcarboxylic acid ester.

25. The process of claim 15, and further comprising hydrolyzing the 3,4′- and/or 4,4′-biphenyldicarboxylic diester separated in (c2) to produce biphenyldicarboxylic acid.
